(19) United States
(12) Patent Application Publication
(10) Pub. No.: US 2025/0177196 A1
(43) Pub. Date: Jun. 5, 2025

(54) Thermoelectric Heating/Cooling Pad

(71) Applicant: C. R. Bard, Inc., Franklin Lakes, NJ (US)

(72) Inventors: Jeffrey Bartolovitch, Boulder, CO (US); Michael R. Hoglund, Windsor, CO (US); Jesse Smith, Broomfield, CO (US)

(21) Appl. No.: 18/844,807

(22) PCT Filed: Apr. 18, 2022
(86) PCT No.: PCT/US2022/025222

§ 371 Date: Sep. 6, 2024

(51) Int. Cl. A61F 7/00; A61F 7/02; G16H 40/63

(52) U.S. Cl. CPC A61F 7/007; A61F 7/0085; A61F 7/02; G16H 40/63; A61F 2007/0054; A61F 2007/0077; A61F 2007/0086; A61F 2007/0093; A61F 2007/0096

(57) ABSTRACT

Disclosed herein are compact systems for performing targeted temperature management including one or more thermal pad assemblies, each or which includes a thermoelectric device. The thermoelectric device heats or cools a thermal exchange fluid circulating within the thermal contact pad to warm or cool a patient. The thermoelectric device may be included in a temperature control module directly attached to the pad. A modularized temperature control module may selectively include one or more thermoelectric devices coupled with a heat exchanger. Logic stored in memory may, via execution of one or more processors, control the temperature of the thermal exchange fluid by defining a polarity and magnitude of an electrical power supplied to the thermoelectric device. The temperature control module may be selectively attached to and detached from the pad. A pre-use unassembled state of the thermal pad assembly include the pad having the thermal exchanged fluid sealed therein.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

TTM systems circulate a fluid (e.g., water) through one or more thermal contact pads coupled with a patient to affect surface-to-surface thermal energy exchange with the patient. In general, TTM systems comprise a TTM fluid control module coupled with at least one contact pad via a fluid deliver line. One such thermal contact pad and related system are disclosed in U.S. Pat. No. 6,197,045 titled “Cooling/heating Pad and System” filed Jan. 4, 1999, which is incorporated herein by reference in its entirety.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

Some TTM systems circulate a fluid (e.g., water) through one or more thermal contact pads coupled to a patient to affect surface-to-surface thermal energy exchange with the patient. In general, TTM systems comprise a TTM fluid control module coupled to at least one contact pad via a fluid deliver line. Such TTM systems include relatively large floor-standing capital equipment that delivers the fluid to the pads via a lengthy (a few meters long) fluid delivery line. Consequently, the size of typical TTM systems require significant room space to accommodate the TTM system. As such, typical TTM systems are incompatible for use within smaller rooms or where the presence of other medical equipment occupy space, such as ICUs, MRI rooms, etc. Furthermore, the typically large TTM systems are incompatible for use in emergency care situations, such as in an ambulance or on an airplane, for example.

In summary, there is a need for TTM systems that are compact, easily transportable, and otherwise compatible for the performance of a TTM therapy a typical patient care facility.

### SUMMARY OF THE INVENTION

Briefly summarized, disclosed herein is a medical pad assembly for exchanging thermal energy with a patient. According to some embodiments, the assembly includes a thermal contact pad configured for placement on the patient, where the pad includes a fluid compartment configured for circulation of a thermal exchange (TE) fluid therein. The assembly further includes a temperature control (TC) module attached to the pad. The TC module includes (i) a pump configured to circulate the TE fluid between the TC module and the fluid compartment and (ii) a thermoelectric device (TED) configured to exchange thermal energy with the TE fluid. In some embodiments, the TC module is attached to the pad along a top side of the pad.

In some embodiments, the TC module includes: (i) a heat exchanger operatively coupled with the TED, where the heat exchanger is configured to facilitate thermal energy transfer between a first side of the TED and the TE fluid; and (ii) a heat sink operatively coupled with the TED a second side of TED, where the heat sink is configured to facilitate thermal energy transfer between the second side of TED and the environment, and where the second side is disposed opposite the first side.

In some embodiments, the TC module includes a power source configured to convert a facility level alternating current (AC) voltage to a direct current (DC) voltage consistent with operation of the TED. In some embodiments, the power source may be spaced away from the TC module so that the AC voltage is spaced away from the TC module during use.

In some embodiments, the TC module includes a temperature controller coupled with the TED, where the temperature controller is configured to cause the TE fluid to increase or decrease the temperature of the TE fluid.

In some embodiments, the TC module includes a temperature sensor operatively coupled with the TE fluid and the temperature controller, and the temperature controller is configured to regulate the temperature of the TE fluid in accordance with a target temperature of the TE fluid stored in a memory of the temperature controller.

In some embodiments, the TC module includes a number of safety temperature sensors operatively coupled with one or more of the TE fluid, the heat exchanger, the TED, the heat sink, or the pump, and the controller is configured to (i) compare a temperature measurement of the number of safety temperature sensors with a temperature safety limit stored in the memory, and (ii) alter an electrical power supplied to the TED when the temperature measurement exceeds the temperature safety limit.

In some embodiments, the TC module is attached to the pad so that an outlet port of the heat exchanger is directly coupled with an inlet port of the fluid compartment and so that an outlet port of the fluid compartment is directly coupled with an inlet port of the pump.

In some embodiments, the TC module is configured for selective attachment to and detachment from the pad.

In some embodiments, the TC module includes a latch to selectively lock the TC module to the pad and unlock the TC module from the pad.

In some embodiments, the heat exchanger and the fluid compartment define a closed fluid volume.

In some embodiments, the pad is one of a number of thermal contact pads having different shapes and/or sizes, and the TC module is configured to attach singly to any one of the number of thermal contact pads.

In some embodiments, the assembly is transitionable from a pre-use unassembled state to an assembled state, where the TC module is separated from the pad in the pre-use unassembled state and the TC module is attached to the pad to an assembled state, where. In the pre-use unassembled state, the pad sealably contains the TE fluid within the fluid compartment.

In some embodiments, the heat exchanger is unprimed with TE fluid in the pre-use unassembled state, and the TE fluid flows from the fluid compartment into the heat exchanger to prime the heat exchanger upon attachment of the TC module to the pad. In some embodiments, the TE fluid is pressurized within the fluid compartment in the pre-use unassembled state.

Also disclosed herein is a medical system for exchanging thermal energy with a patient. According to some embodiments, the medical system includes one or more medical pad assemblies disclosed above. The system further includes a system module communicatively coupled with the one or more medical pad assemblies, where the system module is configured to define an operating configuration of each of the one or more medical pad assemblies.

In some embodiments, defining the operating configuration includes configuring the one or more medical pad assemblies to warm or cool the patient.

In some embodiments, the system module is communicatively coupled with a body temperature measurement device, and defining the operating configuration includes configuring the one or more medical pad assemblies to warm or cool the body temperature toward a target body temperature stored in a memory of the system module.

In some embodiments, the system module is wirelessly coupled with the one or more medical pad assemblies.

Also disclosed herein is a medical system for exchanging thermal energy with a patient that includes: a thermal contact pad configured for placement on the patient, the pad including a fluid compartment configured for circulation of a thermal exchange (TE) fluid therein; and a system module. The system module includes: (i) a heat exchanger including a number of fluid channels, the heat exchanger coupled with the pad via a fluid delivery line; (ii) a pump configured to circulate the TE fluid between the heat exchanger and the pad; (iii) a temperature control (TC) module operatively coupled with the heat exchanger, the TC module configured to warm and/or cool the TE fluid circulating within the heat exchanger; and (iv) a console including one or more processors and a non-transitory computer-readable medium having stored thereon logic that, when executed by the one or more processors, causes operations of the system that include defining a temperature of the TE fluid.

In some embodiments, the heat exchanger is configured to couple with more than one TC module. In some embodiments, the system module includes at least two TC modules operatively coupled with the heat exchanger.

In some embodiments, the heat exchanger is configured to couple with one or more TC modules along a first side of the heat exchanger. The heat exchanger is further configured to couple with one or more TC modules along a second side of the heat exchanger, where the second side is disposed opposite the first side.

In some embodiments, the TC module includes: a thermoelectric device (TED) thermally coupled with the heat exchanger along a first side of the TED; and a heat sink thermally coupled with the TED along a second side of the TED, where the second side is disposed opposite the first side.

In some embodiments, the operations include switching a polarity of an electrical power supplied to the TED between a first polarity and an opposite second polarity. The first polarity causes the TED to supply thermal energy to the heat exchanger to increase a temperature of the TE fluid circulating with the heat exchanger, and the second polarity causes the TED to extract thermal energy from the heat exchanger to decrease the temperature of the TE fluid circulating with the heat exchanger.

In some embodiments, the system module includes an outlet temperature sensor operatively coupled with the TE fluid exiting the heat exchanger, where the outlet temperature sensor is configured to provide an electrical signal to the console in accordance with a temperature of the TE fluid exiting the heat exchanger, and the operations include: (i) comparing the temperature of the TE fluid exiting the heat exchanger with a target temperature for the TE fluid stored in the memory; and (ii) as a result of the comparison, adjusting an electrical power supplied to the TED to move the temperature of the TE fluid exiting the heat exchanger toward the target temperature.

In some embodiments, the system module further includes an inlet temperature sensor operatively coupled with the TE fluid entering the heat exchanger, where the input temperature sensor is configured to provide an electrical signal to the console in accordance with a temperature of the TE fluid entering the heat exchanger, and the operations include: (i) determining a temperature difference between the TE fluid exiting the heat exchanger and the TE fluid entering the heat exchanger; and (ii) adjusting the electrical power supplied to the TED based on the temperature difference.

In some embodiments, the system module further includes an ambient temperature sensor configured to provide an electrical signal to the console in accordance with an ambient temperature of the patient environment, and the operations include adjusting the electrical power supplied to the TED based on the ambient temperature.

In some embodiments, the system module further includes a safety temperature sensor operatively coupled with the TE fluid exiting the heat exchanger, where the safety temperature sensor is configured to provide an electrical signal to the console in accordance with a temperature of the TE fluid exiting the heat exchanger, and the operations include: (i) comparing the temperature of the TE fluid exiting the heat exchanger with a safety temperature limit stored in a memory of the system module; and (ii) as a result of the comparison, altering the electrical power supplied to the TED when the temperature of TE fluid exiting the heat exchanger exceeds the safety temperature limit.

In some embodiments, the pump is hydraulicly positioned between the pad and the heat exchanger, where the pump is configured to cause TE fluid to flow from the pad to the heat exchanger. A pressure sensor is hydraulicly positioned between the pad and the pump, where the pressure sensor is configured to provide an electrical signal to the console in accordance with a pressure of the TE fluid within the pad, and the operations include: (i) comparing the pressure of the TE fluid within the pad with a pressure limit stored in the memory; and (ii) as a result of the comparison, adjusting an electrical power supplied to the pump when the pressure of the TE fluid within the pad exceeds the pressure limit.

In some embodiments, a power source of the system includes a battery.

Also disclosed herein is a method performed by a medical system for exchanging thermal energy with a patient. According to some embodiments, the method includes circulating a thermal exchange (TE) fluid between a heat exchanger and thermal contact pad applied to the patient. The method further includes providing an electrical power via a power module of the system to a thermoelectric device (TED) operatively coupled with the heat exchanger to define a thermal energy exchange between the TED and the TE fluid circulating with the heat exchanger, and the power module is configured to define a polarity of the electrical power supplied to the TED to cause the TED to extract thermal energy from or supply thermal energy to the TE fluid circulating within the heat exchanger to cool or warm the patient.

In some embodiments of the method, the power source is configured to selectively define the polarity of the electrical power supplied to the TED between a first polarity and an opposite second polarity, where the electrical power at the first polarity causes the TED to extract thermal energy from the TE fluid circulating within the heat exchanger, and where the electrical power at the second polarity causes the TED to provide thermal energy to the TE fluid circulating within the heat exchanger to warm the patient.

In some embodiments, the method further includes: (i) determining a temperature of the TE fluid circulating within the thermal contact pad, and (ii) adjusting the electrical power to move the temperature of the TE fluid toward a target temperature for the TE fluid stored in a memory of the system.

In some embodiments, the method further includes: (i) receiving a body temperature signal from a body temperature measurement device, (ii) adjusting the target temperature for the TE fluid to move the body temperature toward a target body temperature stored in the memory.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a targeted temperature management system (TTM) system coupled with a patient, in accordance with some embodiments;

FIG. 2 is a block diagram of a console of a system module of the TTM system of FIG. 1, in accordance with some embodiments;

FIG. 3 illustrates a thermal contact pad assembly of the TTM system of FIG. 1, in accordance with some embodiments;

FIG. 4 is a side view illustration of the thermal contact pad assembly of the TTM system of FIG. 3, in accordance with some embodiments;

FIG. 5 is a block diagram of a controller of a thermal contact pad assembly of FIG. 3, in accordance with some embodiments;

FIG. 6 is a cross-sectional side view illustration of a portion of the thermal contact pad assembly of FIG. 3 in a pre-use unassembled state, in accordance with some embodiments;

FIG. 7A illustrates another embodiment of a targeted temperature management system (TTM) system, in accordance with some embodiments;

FIG. 7B illustrates a functional schematic of the TTM system of FIG. 7A, in accordance with some embodiments;

FIG. 8 is a block diagram of a controller of TTM system of FIGS. 7A-7B, in accordance with some embodiments;

FIG. 9 illustrates a further embodiment of a targeted temperature management system (TTM) system, in accordance with some embodiments;

FIG. 10A illustrates a temperature control module that may be employed with the TTM systems of FIGS. 7A or 9, in accordance with some embodiments;

FIG. 10B illustrates a top perspective view the TC sub-assembly of the temperature control module of FIG. 10A, in accordance with some embodiments;

FIG. 10C illustrates an exemplary arrangement of the TC assemblies coupled with a heat exchanger of the temperature control module of FIG. 10A, in accordance with some embodiments;

FIG. 11 illustrates thermoelectric device of the TC sub-assembly of FIG. 10B, in accordance with some embodiments; and

FIG. 12 illustrates an extendable heat exchanger that may be employed with the temperature control module of FIG. 10A, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to,” “coupled with,” and “in communication with” refer to any form of interaction between two or more entities, including but not limited to mechanical, electrical, thermal, and fluid interaction. Two components may be coupled with each other even though they are not in direct contact with each other. For example, two components may be coupled to each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Unless defined otherwise, all technical and scientific terms used herein have the same meaning as commonly understood by those of ordinary skill in the art.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

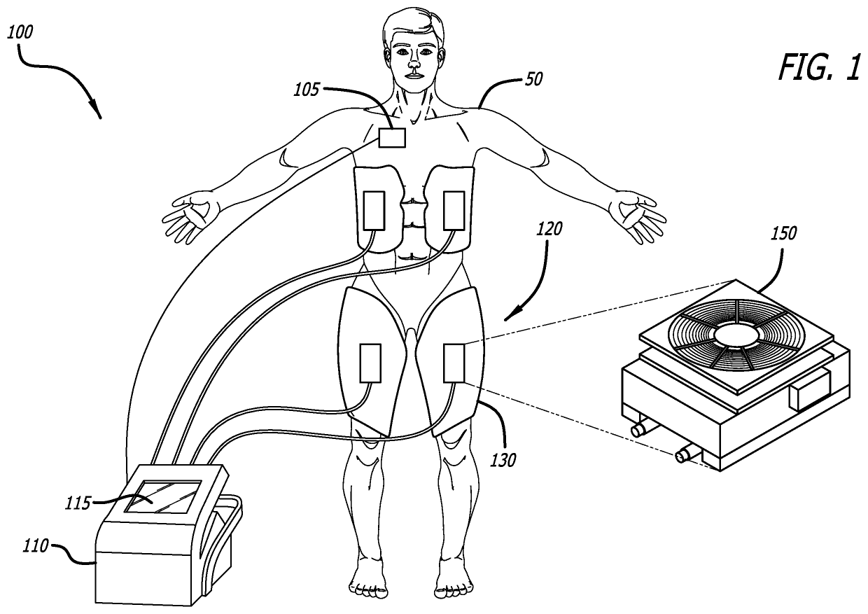

FIG. 1 illustrates a targeted temperature management (TTM) system 100 (e.g.,) in use with a patient 50. The TTM system (system) 100 may be used to cool and/or warm the patient 50 in accordance with a TTM therapy. The system 100 generally includes a system module 110 communicatively coupled with a number (e.g., 1, 2, 3, 4 or more) thermal contact pad assemblies 120 applied to a patient 50. The system module 110 may be communicatively coupled with each pad assembly 120 via a wired connection or the system module 110 may be wirelessly coupled with each pad assembly 120. The system module 110 may also include a user interface 115 to facilitate input of system information and data. The user interface 115 may also portray information and/data on a screen of the user interface.

The thermal contact pad assembly 120 generally includes a temperature control (TC) module 150 attached to a thermal contact pad (pad) 130. The pad 130 is applied to the patient 50 so that a bottom side of the pad 130 is thermally coupled with the patient 50 and the (TC) module is attached to pad 130 along an opposite top side of the pad 130.

The system module 110 generally defines the operating configuration of each of the pad assemblies 120. More specifically the system module 110 configures each pad assembly 120 to either cool or warm the patient 50.

In some embodiments, the system module 110 may be communicatively coupled body temperature measurement device 105. The body temperature measurement device 105 is coupled to the patient 50 to indicate a core body temperature of the patient 50. In some instances, the body temperature measurement device 105 may be an esophageal, bladder catheter, or rectal temperature sensor. In use, the system module 110 may acquire body temperature data from the body temperature measurement device 105 and configure each pad assembly 120 to move (i.e., cool or warm) the body temperature of the patient 50 toward a target body temperature as defined by the system module 110. The system module 110 may be coupled body temperature measurement device 105 wirelessly or via a wired connection.

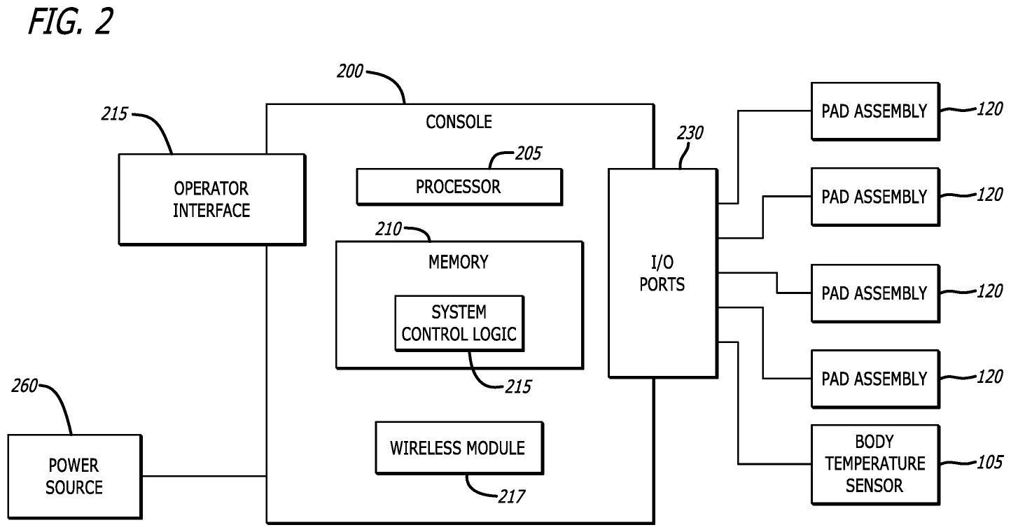

FIG. 2 illustrates a block diagram of a console 200 of the system module 110 of FIG. 1. The console 200 includes a processor 205 and memory 210 including a non-transitory, computer-readable storage medium, and the memory 210 includes system control logic 215 stored thereon. The console 200 includes, and input/output (I/O) ports 230 to facilitate communication with the one or more pad assemblies 120 via a wired connection. The console may optionally include wireless module 217 to facilitate wireless communication with the pad assemblies 120. The I/O ports 230 may also facilitate communication with the body temperature measurement device 105 via a wired connection. The optional wireless module 217 may also facilitate wireless communication with the body temperature measurement device 105. The console 200 is coupled with a power source 260 which may include a facility power or a battery. The console 200 is coupled with the user interface 115 of the system module 110.

The system control logic 215 may control the operation of the pad assemblies 120. The system control logic 215 may communicate a target pad temperature to the pad assembly 120. The system control logic 215 may receive patient temperature data from the body temperature measurement device 105 indicating the current temperature of the patient 50. The system control logic 215 may compare the patient temperature data with the target patient temperature, and as a result of the comparison, the system control logic 215 may modify the target pad temperature and communicate the revised target pad temperature to the pad assemblies 120. The system control logic 215 may further enable and/or disable operation of any or all of the pad assemblies 120.

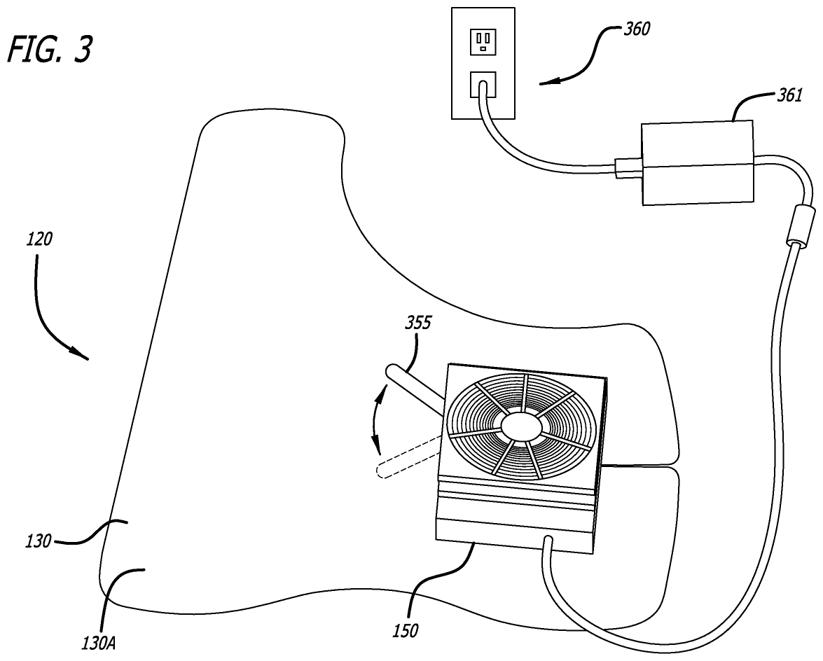

FIG. 3 illustrates the pad assembly 120. As stated above, the pad assembly 120 includes the TC module 150 attached to the pad 130 on the top side 130A of the pad 130. The TC module 150 includes a power source 360. In some embodiments, the power source 360 may be an electrical power system of a facility. In other embodiments, the power source 360 may be a portable power source such as a battery pack or a generator. The power source 360 may include a power converter 361 that converts a facility-level alternating current (AC) voltage to a direct current (DC) voltage. The DC voltage may be relatively low (e.g., less than 24 volts) consistent with operation of the TC module 150. The DC voltage less than 24 volts may also prevent an electrical hazard to the patient 50 during use. In some embodiments, the power converter 361 may be spaced away (e.g., 1, 2 or more meters) from the TC module 150 or the patient 50 so that the facility-level AC voltage is separated from the TC module 150 or patient 50 a safe distance.

The TC module 150 may include a latching mechanism 355 to facilitate selective locking of the TC module 150 to and unlocking of the TC module 150 from the pad 130. The latching mechanism 355 may include any suitable components and functionality to (i) prevent separation of the TC module 150 from the pad 130 when the latching mechanism 355 is disposed in a locked state and (ii) allow separation of the TC module 150 from the pad 130 when the latching mechanism 355 is disposed in an unlocked state.

In some embodiments, the pad assembly 120 may be configured to facilitate thermal energy exchange with the patient 50 independent from the system module 110. As such, in some embodiments, one or more pad assemblies 120 may define the system 100. In other words, the system module 110 may be omitted.

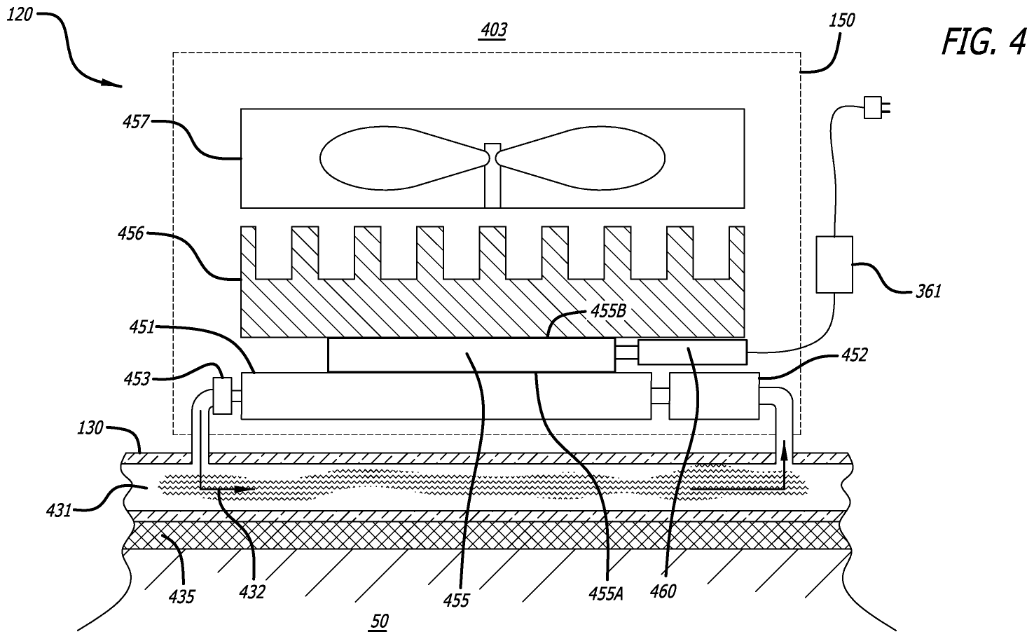

FIG. 4 is a cross-sectional illustration of the pad assembly 120 showing the TC module 150 attached to the pad 130. The pad assembly 120 is generally configured to define a thermal energy exchange with the patient 50. The TC module 150 is configured to heat and/or cool a thermal exchange (TE) fluid 432. A pump 452 of the TC module 150 circulates the TE fluid 432 (e.g., water) between the TC module 150 and the pad 130 to warm/cool the patient 50. The pad 130 includes a fluid compartment 431 extending across the pad 130, and the TE fluid 432 circulates within the fluid compartment 431. The pad 130 may further include a hydrogel layer 435 disposed across a bottom side the pad 130 so as to be positioned between the pad 130 and the patient 50 during use.

The TC module 150 includes a thermoelectric device (TED) 455 thermally coupled between a heat exchanger 451 and a heat sink 456, and the TED 455 defines a thermal energy exchange between the heat exchanger 451 and the heat sink 456 in accordance with an electrical power supplied to the TED 455. The heat exchanger 451 transfers thermal energy between the TED 455 and the TE fluid 432 circulating through the heat exchanger 451. The heat sink 455 transfers thermal energy from the TED 455 to the environment 403. A fan 457 enhances thermal energy transfer from the heat sink 455 to the environment 403 via forced convection. The heat exchanger 451 may be formed of aluminum or any other suitable material consistent with conduction of thermal energy.

The TEC module 150 is configured to operate in a first mode of operation to cool the patient 50 and a second mode of operation to warm the patient 50. More specifically the TEC module 150 is configured to supply an electrical power at a first polarity to the TED 455 in the first mode and supply the electrical power to the TED 455 at a opposite second polarity to the TED 455 in the second mode.

In the first mode, the electrical power supplied to the TED 455 at the first polarity causes a first temperature difference between a first side 455A and a opposite second side 455B, where a temperature of the first side 455A is less than a temperature of the second side 455B. As the first side 455A is coupled with the heat exchanger 451 and the second side 455B is coupled with the heat sink 456, the TED 455 transfers thermal energy from the heat exchanger 451 to the heat sink 457 to cool the TE fluid 432 within the heat exchanger 451 and thereby, cool the patient 50.

In the second mode, the electrical power supplied to the TED 455 at the second polarity causes a second temperature difference between a first side 455A and the second side 455B, where the temperature of the first side 455A is greater than the temperature of the second side 455B. As such, in the second mode of operation, the TED 455 transfers thermal energy from the heat sink 456 to the heat exchanger 451 to warm the TE fluid 432 within the heat exchanger 451 and thereby, warm the patient 50.

The TC module 150 includes a controller 460 generally configured to regulate the thermal energy exchange with the patient 50. As such, the controller 460 is configured to define the electrical power supplied to the TED 455. The controller 460 may define (i) the polarity of the electrically powered supplied to the TED 455 and (ii) a magnitude of the voltage of the electrical power supplied to the TED 455.

In some embodiments, the TC module 150 may include a temperature sensor 453 thermally coupled with the TE fluid 432. The temperature sensor 453 may be positioned so as to measure a temperature of the TE fluid 432 entering the fluid compartment 431. The temperature sensor 453 is electrically coupled with the controller 460.

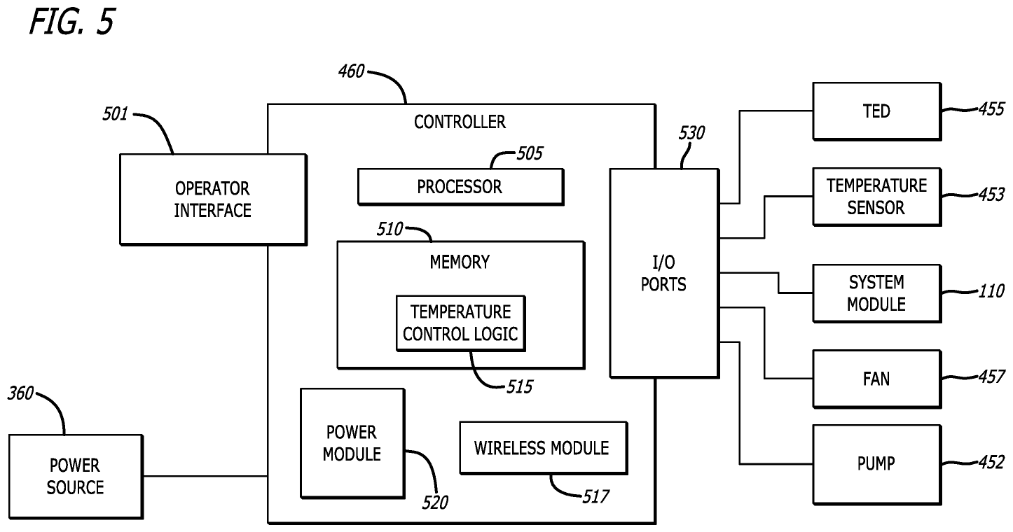

FIG. 5 illustrates a block diagram of the controller 460 of FIG. 4, in accordance with some embodiments. The controller 460 includes a processor 505 and memory 510 including a non-transitory, computer-readable storage medium, and the memory 510 includes temperature control logic 515 stored thereon. The controller 460 further includes a power module 520 and input/output (I/O) ports 530. The controller 460 receives electrical power from the power source 360. The power module 520 may be controllable via the temperature control logic 515 when executed by the processor 505. The I/O ports 530 facilitate connection of the controller 460 with one or more of the TED 455, the temperature sensor 453, the system module 110, the fan 457, or the pump 452.

The TC module 150 may optionally include a user interface 501 coupled to the controller 460. The user interface 501 may be communicatively coupled with the controller 460 via a wired or wireless connection. In some embodiments, the user interface 501 may be included with the controller 460. In other embodiments, the user interface 501 may be operated via an external device such as a network computer, a tablet, or a cell phone. The user interface 501 may include a graphical user interface (GUI). The user interface 501 may facilitate input and/or display of operational settings such as a target temperature for the pad 130 (i.e., the TE fluid 432 circulating with the fluid compartment 431), for example.

The controller 460 may optionally include a wireless module 517 to facilitate wireless communication with devices external of controller 460. The external devices may include a facility network, the user interface 501, the system module 110, or any other device that may be used in accordance with the TTM therapy.

The temperature control logic 515 is configured to perform operations of the TC module 150. The operations may include defining the polarity of the electrical power supplied to the TED 455. The operations may further include defining a magnitude of the electrical power supplied to the TED 455. In some embodiments, the logic 515 may define a fixed electrical power supplied to the TED 455. In some embodiments, logic 515 may (i) receive temperature data from the temperature sensor 453 and (ii) alter the electrical power (e.g., the polarity and/or magnitude) supplied to the TED 455 based on the temperature data. In some embodiments, the logic 515 may (i) compare the temperature of the TE fluid 432 (i.e., the temperature indicated by temperature data acquired from the temperature sensor 453) with a target pad temperature stored in memory and (ii) as a result of the comparison define the electrical power supplied to the TED 455 to move the temperature of the TE fluid 432 toward the target pad temperature.

In some embodiments, the logic 515 may (i) receive a target body temperature from the system module 110, and (ii) define the electrical power supplied to the TED 455 to cool or warm the patient 50 toward the target body temperature. In some embodiments, the logic 515 may (i) receive a target pad temperature from the system module 110, and (ii) override the target pad temperature stored in memory with the target pad temperature received from the system module 110.

The pad assembly 120 may be configured for use with varying patient sizes and shapes. Furthermore, a shape of the pad 130 may be configured for placement at one of a number of placement locations on a patient 50. As such, in some embodiments, the TC module 150 may be coupled with (i.e., attached to and/or locked with) any one of a number of pads 130 to define the pad assembly 120.

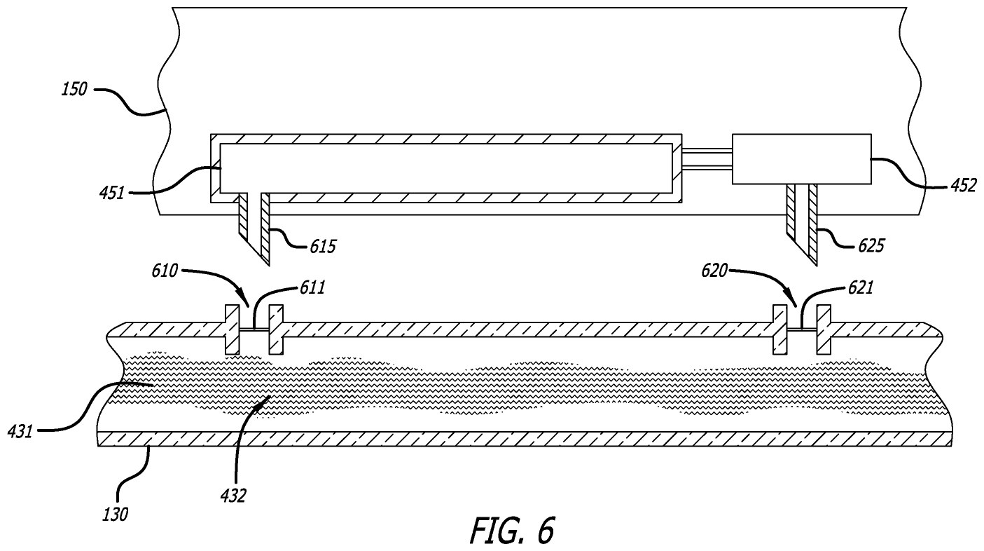

FIG. 6 is a cross sectional illustration of portions of the TC module 150 unattached to the pad 130. The pad assembly 120 may be configured to transition from a pre- use unassembled state to an assembled state. The TC module 150 is separated from the pad 130 in the pre-use unassembled state and the TC module 150 is attached to the pad 130 to an assembled state. In the pre-use unassembled state, the pad 130 sealably contains the TE fluid 432 within the fluid compartment 431, and the heat exchanger 452 is unprimed with the TE fluid 432. Upon attachment of the TC module 150 to the pad 130, the TE fluid 432 may flow from the fluid compartment 431 into the heat exchanger 451 to prime the heat exchanger 451. Although not required, the TE fluid 432 may be pressurized within the fluid compartment 431 in the pre-use unassembled state.

According to one exemplary implementation, the pad 130 (or more specifically the fluid compartment 431) may include an inlet port 610 and an outlet port 620. The inlet port 610 includes a frangible septum 611 sealing the inlet port 610, and the outlet port 620 includes a frangible septum 621 sealing the outlet port 620. Correspondingly, the TC module 150 includes an outlet spike 615 in fluid communication with the heat exchanger 451 and an inlet spike in fluid communication with the pump 452. Upon attachment of the TC module 150 with pad 130 (i.e., upon the pad assembly 120 transitioning from the pre-use unassembled state to the assembled state), the outlet spike 615 pierces the septum 611 and a defines a seal with the inlet port 610. Similarly, upon attachment of the TC module 150 with pad 130, the inlet spike 625 pierces the septum 621 and a defines a seal with the outlet port 620. After attachment, the TE fluid 432 may flow from the fluid compartment 431 into the heat exchanger 451, thereby priming the heat exchanger 451.

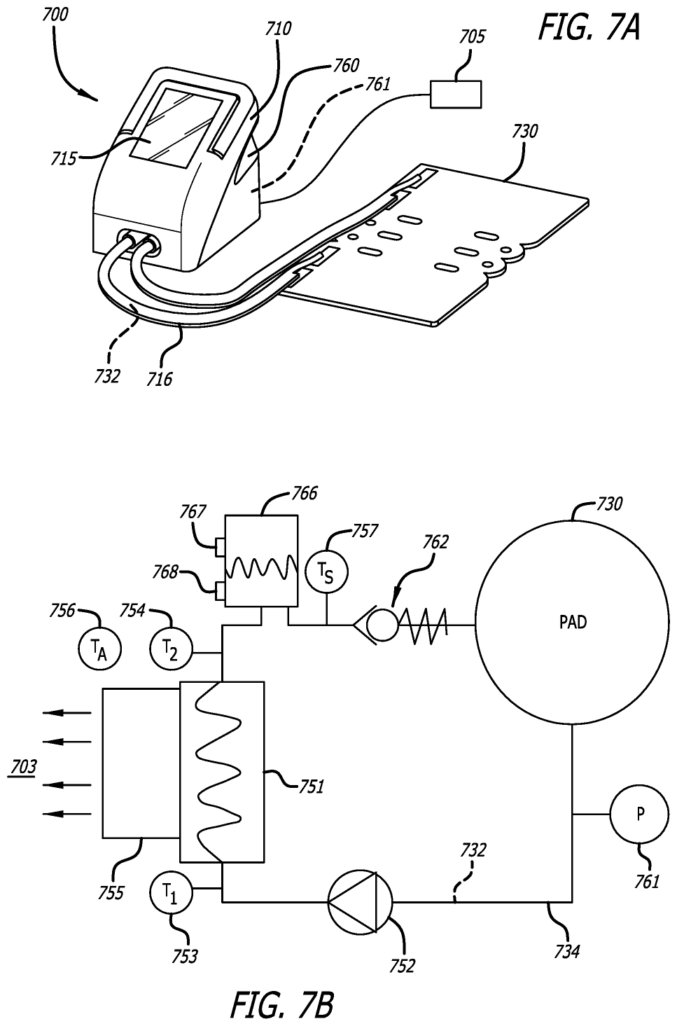

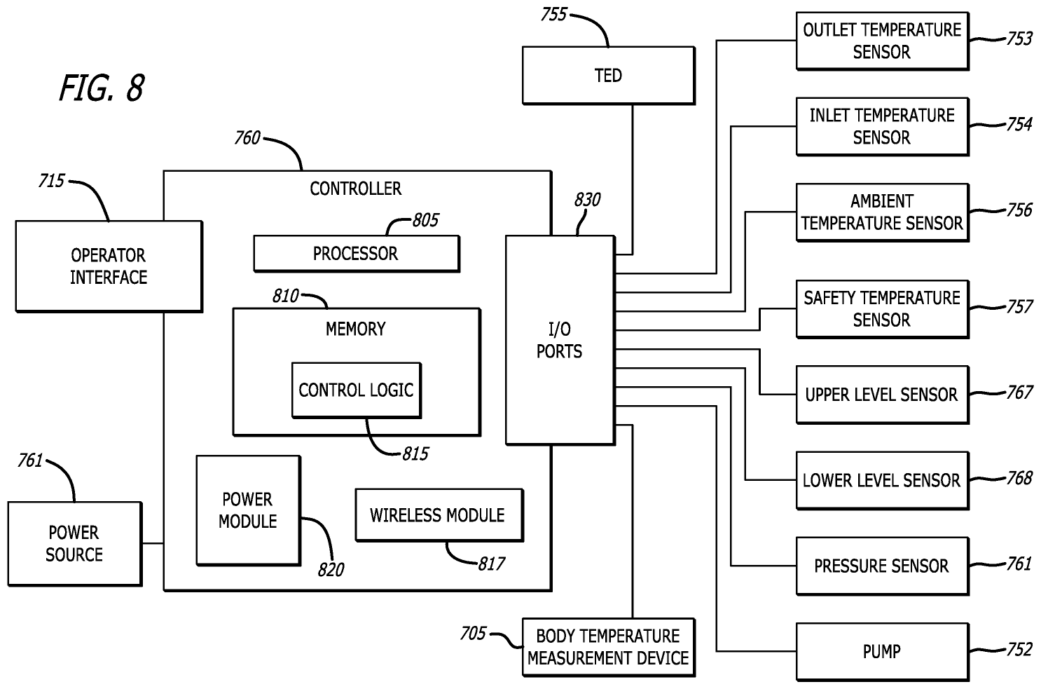

FIGS. 7A-8 illustrate a TTM system 700 that can, in certain respects, resemble components of the TTM system 100 described in connection with FIGS. 1-6. It will be appreciated that all the illustrated embodiments may have analogous features. Relevant disclosure set forth above regarding similarly identified features thus may not be repeated hereafter. Moreover, specific features of the TTM system 100 and related components shown in FIGS. 1-6 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the TTM system of FIGS. 7A-8. Any suitable combination of the features, and variations of the same, described with respect to the TTM system and components illustrated in FIGS. 1-6 can be employed with the TTM system and components of FIGS. 7A-8, and vice versa.

FIG. 7A is a perspective illustration of the TTM system 700 that is configured to cool or warm a patient similar to the TTM system 100. The TTM system 700 generally includes a system module 710 fluidly coupled with a thermal contact pad 730 via a fluid deliver line 716 having a TE fluid 732 flowing therethrough to and from the pad 730. The system module 710 defines a temperature of the TE fluid 732 that is circulated within the pad 730 to define a thermal energy exchange with the patient (see FIG. 1) to cool or warm the patient. The system 700 may be configured for use within a number of different environments, such as a healthcare facility, outside, within a helicopter, or within a fixed wing aircraft, for example. As such, the environmental conditions may include extended ranges beyond the conditions of a typical healthcare facility. Exemplary environmental conditions may, without limitation, include temperature, pressure, motion, and orientation. Various embodiments of the system 700 may include one of a number of pads 730, where the number of pads 730 include different sizes and/or shapes.

The system module 710 may include a user interface 715 to facilitate input of system information and data. The user interface 715 may also portray information and/data on a screen of the user interface. A power source 761 provides electrical power to system module 710, and the power source 761 may be a battery. The user interface 715 may include a graphical user interface (GUI). The user interface 715 may facilitate input of operational settings, such as a target temperature for the pad 730 (i.e., the TE fluid 732 circulating with the pad 730), for example. The user interface 715 may also include a screen for portraying operational information or data. The system module 710 may be coupled with a body temperature measurement device 705 which may resemble the body temperature measurement device 105 of FIG. 1.

FIG. 7B illustrates a functional schematic of the system 700. As shown, the system includes the TE fluid 732 circulating along a fluid flow path 734 between the pad 730 and a heat exchanger 751. A thermoelectric device (TED) 755 defines a thermal energy exchange between the TE fluid 732 within the heat exchanger 751 and the environment 703.

The system 700 include a number of hydraulic components. A pump 752, disposed downstream of the pad 730 and upstream of the heat exchanger 751, defines the flow of TE fluid 732. The preloaded check valve 762 prevents flow toward the pad 730 in the direction of the pump if a pressure of the TE fluid 732 within pad 730 exceeds a pressure limit. In some embodiments, the pressure limit may be atmosphere in pressure (i.e., zero gauge pressure). The preloaded check valve 762 may also prevent flow of TE fluid 732 away from the pad 730 in direction opposite the pump 752.

The system 700 may also include a compensation chamber 766 disposed downstream of the heat exchanger 751 and upstream of the check valve 762. The compensation chamber 766 may be partially filled with the TE fluid 732 so as to define an air volume above the level of TE fluid 732 within the compensation chamber 766. In the illustrated embodiment, the compensation chamber 766 is closed at the top to define a closed fluid flow path 734. In alternative embodiments, the compensation chamber 766 may be vented to the environment so as to define an environmental pressure reference for the fluid flow path 734. The compensation chamber 766 may also be configured to remove air bubbles from TE fluid 732 circulating along the fluid flow path 734.

The system 700 may also include various sensors to provide data to the controller 760. The system 700 may include a pressure sensor 761 disposed downstream of the pad 730 and upstream of the pump 752, and as such, the pressure sensor 761 may indicate the pressure of the TE fluid 732 within the pad 730. The compensation chamber 766 includes upper and lower fluid level sensors 767, 768, respectively.

The system 700 includes an output temperature sensor 754 configured and positioned to indicate a temperature of the TE fluid 732 exiting the heat exchanger 751 and entering the pad 730. In some embodiments, the system 700 may also include an input temperature sensor 753 configured and positioned to indicate a temperature of the TE fluid 732 exiting the pad 730 and entering the heat exchanger 751. In the illustrated embodiment, the system 700 may also include a safety temperature sensor 757 configured and positioned to indicate a temperature of the TE fluid 732 entering the pad 730. In other embodiments, the system 700 may include one or more additional safety temperatures thermally coupled with the heat exchanger, the TED, the heat sink, or the pump. The system 700 may optionally include an ambient temperature sensor 756 configured and positioned to indicate a temperature of the environmental 703.

FIG. 8 illustrates a block diagram of the controller 760 of FIG. 7A, in accordance with some embodiments. The controller 760 includes a processor 805 and memory 810 including a non-transitory, computer-readable storage medium, and the memory 810 includes control logic 815 stored thereon. The controller 760 further includes a power module 820 and input/output (I/O) ports 830. The power module 820 may be controllable via the control logic 815 when executed by the processor 805. The I/O ports 830 facilitate connection of the controller 760 with one or more of the TED 755, the outlet temperature sensor 754, the inlet temperature sensor 753, the ambient temperature sensor 756, the safety temperature sensor 757, the upper level sensor 767, the lower level sensor 768, the pressure sensor 761, the pump 752, and the body temperature measurement device 705. The controller 760 receives electrical power from the power source 761.

The controller 760 is coupled with the user interface 715. In some embodiments, the user interface 715 may be included with the system module 710. In other embodiments, the user interface 715 may be communicatively coupled with the controller 760 via a wired or wireless connection. In other embodiments, the user interface 715 may be operated via an external device such as a network computer, a tablet, or a cell phone.

The controller 760 may optionally include a wireless module 817 to facilitate wireless communication with devices external of controller 760. The external devices may include a facility network, the user interface 801, the system module 110, or any other device that may be used in accordance with the TTM therapy. Specifically, the wireless module 817 may facilitate communication with the body temperature measurement device 705.

The control logic 815 is configured to perform operations of the system 700. The operations may generally include defining conditions of the TE fluid 732, such as a temperature, flow, and pressure, for example.

The operations may include defining a polarity of an electrical power supplied to the TED 755. The operations may further include defining a magnitude of the electrical power supplied to the TED 755. In some embodiments, the logic 815 may define a fixed electrical power supplied to the TED 755, such as an electrical power to maximize cooling the patient, for example.

The control logic 815 is configured to (i) receive data from each of the temperature sensors, i.e., the outlet temperature sensor 754, the inlet temperature sensor 753, the ambient temperature sensor 756, and the safety temperature sensor 757 sensors, and (ii) perform operations in accordance with the data (i.e., a temperature measurement). In some embodiments, the logic 815 may alter the electrical power (e.g., the polarity and/or magnitude) supplied to the TED 755 based on the temperature exiting the heat exchanger 751 (i.e., entering the pad 730) as acquired by the outlet temperature sensor 754.

In some embodiments, the logic 815 may (i) compare the temperature of the TE fluid 732 exiting the heat exchanger 751 with a target pad temperature stored in memory, and (ii) as a result of the comparison, define the electrical power supplied to the TED 755 to move the temperature of the TE fluid 732 toward the target pad temperature.

In some embodiments, the logic 815 may (i) determine a difference between the temperature of the TE fluid 732 exiting the heat exchanger 751 (the temperature measurement acquired by the outlet temperature sensor 754) and a temperature of the TE fluid 732 entering the heat exchanger 751 (i.e., the temperature measurement acquired by the inlet temperature sensor 753) and (ii) as a result of the determination, define the electrical power supplied to the TED 755 based on the temperature difference. By way of example, the logic 815 may compare the determined temperature difference with a temperature difference stored in the memory 810, and increase the magnitude of the electrical power when determined temperature difference exceeds the temperature difference stored in the memory 810.

In some embodiments, the logic 815 may (i) compare the temperature of the TE fluid 732 entering the pad 730, as acquired by the safety temperature sensor 757, with a temperature safety limit stored in memory, and (ii) as a result of the comparison, alter the electrical power supplied to the TED 755 to move the temperature of the TE fluid 732 entering the pad 730 away from the temperature safety limit. In some embodiments, altering the electrical power may include switching off the electrical power. In some embodiments, the logic 815 may provide an alert to the user when the temperature of the TE fluid 732 entering the pad 730 exceeds the temperature safety limit.

In some embodiments, the logic 815 may (i) compare an ambient temperature measurement of the environment, as acquired by the ambient temperature sensor 756, with an ambient temperature stored in memory, and (ii) as a result of the comparison, alter the electrical power supplied to the TED 755. By way of example, in some embodiments, the ambient temperature stored in memory may be a standard ambient temperature, and the altering the electrical power may include decreasing the magnitude of the electrical power, when ambient temperature measurement is less than the ambient temperature stored in memory.

The control logic 815 is configured to (i) receive data from each of the body temperature measurement device 705, and perform operations in accordance with temperature data received therefrom. The logic 815 may compare body temperature measurement with a target body temperature stored in the memory 810. As a result of the comparison, the logic 815 may alter the electrical power supplied to the TED 755 to move the body temperature (i.e., cool or warm the patient) toward the target body temperature stored in the memory 810.

The logic 815 is configured to receive pressure measurement data from the pressure sensor 761, where the pressure measurement indicates a pressure of the TE fluid 732 within pad 730. As the pad 730 is hydraulically positioned downstream of the preloaded check valve 762 and upstream of the pump 752, the pressure of the TE fluid 732 within the pad 730 as measured by the pressure sensor 761 may typically be negative. The memory 810 may include a typical (e.g., normally expected) pad pressure range stored therein. The logic may (i) compare the pressure measurement with the typical pad pressure range, and (ii) alter an electrical power supplied to the pump when the pressure measurement exceeds the typical pad pressure range (i.e., is beyond an upper or lower limit). By way of example, a pressure measurement that exceeds the typical range on the upper side (toward the positive) may indicate a low flow rate of TE fluid 732. In response, the logic 815 may increase the electrical power to the pump 752 to increase the flow rate. By way of another example, a pressure measurement that exceeds the typical range on the lower side (toward the negative) may indicate a blockage of flow of TE fluid 732 through the pad 730. In response, the logic 815 may switch off the electrical power to the pump 752. The logic 815 may also provide an alert to the user when the pressure measurement that exceeds the typical range.

The logic 815 is configured to receive data from the upper and lower level sensors 767, 768 respectively, of the compensation chamber 766. In the illustrated embodiment, the upper and lower level sensors 767, 768 may be binary sensors that indicate only of the level of TE fluid 732 within the compensation chamber 766 is either above an upper level limit defined the upper level sensor 767 or below a lower level limit defined the lower level sensor 768. In other embodiments, the compensation chamber 766 may include a one or more level sensors capable of proving variable data regarding the level of TE fluid 732.

In some instances, when the level of TE fluid 732 exceeds either the upper level limit or the lower level limit, a corrective action by the user may be warranted. As such, the logic 815 may provide an alert to the user when the level of TE fluid 732 exceeds either the upper level limit or the lower level limit. The logic 815 may also alter the electrical power supplied to the pump 752 when the level of TE fluid 732 exceeds either the upper level limit or the lower level limit. By way of example, in some instances a leak of the TE fluid from the pad 730 (or the system 700 generally) may cause the TE fluid 732 within the compensation chamber 766 to exceed the lower level limit. As such, the logic 815 may switch off the electrical power supplied to the pump 752 to limit the amount of TE fluid 732 leaking from the system 700. By way of another example, in some instances a leak of air into the fluid flow path 734 may cause the TE fluid 732 within the compensation chamber 766 to exceed the upper level limit. As such, the logic 815 may switch off the electrical power supplied to the pump 752 so that the user may resolve the air leak.

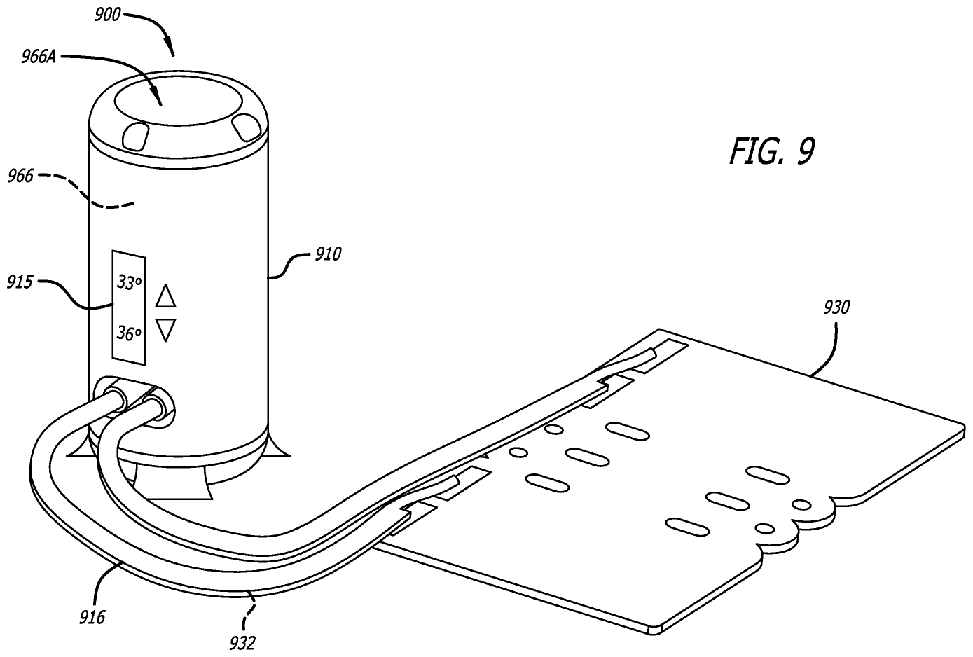

FIG. 9 illustrate another embodiment of a TTM system 900 that can, in certain respects, resemble components of the TTM system 700 described in connection with FIGS. 7A-8. It will be appreciated that all the illustrated embodiments may have analogous features. Accordingly, like features are designated with like reference numerals, with the leading digits incremented to “9.” For instance, the pad is designated as “730” in FIGS. 7A-8, and an analogous pad is designated as “930” in FIG. 9. Relevant disclosure set forth above regarding similarly identified features thus may not be repeated hereafter. Moreover, specific features of the TTM system 700 and related components shown in FIGS. 7A-8 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the TTM system 900 of FIG. 9. Any suitable combination of the features, and variations of the same, described with respect to the TTM system and components illustrated in FIGS. 7A-8 can be employed with the TTM system and components of FIG. 9, and vice versa.

FIG. 9 is a perspective illustration of the TTM system 900 that is configured to cool a patient. The TTM system 900 generally includes a system module 910 fluidly coupled with a thermal contact pad 930 via a fluid deliver line 916 having a TE fluid 932 flowing therethrough to and from the pad 930. The system module 910 defines a temperature of the TE fluid 932 that is circulated within the pad 930 to define a thermal energy exchange with the patient (see FIG. 1) to cool the patient. The system 900 may be configured for use within a number of different environments, such as a healthcare facility, outside, within a helicopter, or within a fixed wing aircraft, for example. As such, the environmental conditions may include extended ranges beyond the conditions of a typical healthcare facility. Exemplary environmental conditions may, without limitation, include temperature, pressure, motion, and orientation. Various embodiments of the system 900 may include one of a number of pads 930, where the number of pads 930 include different sizes and/or shapes. In some embodiments, the system 900 may be configured to warm the patient, and in further embodiments, the system 900 may be configured to selectively cool or warm the patient.

The system module 910 may include a user interface 915 to facilitate input or adjustment of a target temperature for the TE fluid 932. In some embodiments, a range for the target temperature may extend between 33° C. and 36° C., inclusive.

The system module 910 includes a compensation chamber 966 containing the TE fluid 932. The compensation chamber 966 includes an open or openable top end 966A. The top end 966A may provide for the addition and/or removal of the TE fluid 932. The top end 966A may also define an atmospheric pressure reference for the TE fluid 932 flowing to and/or from the pad 930.

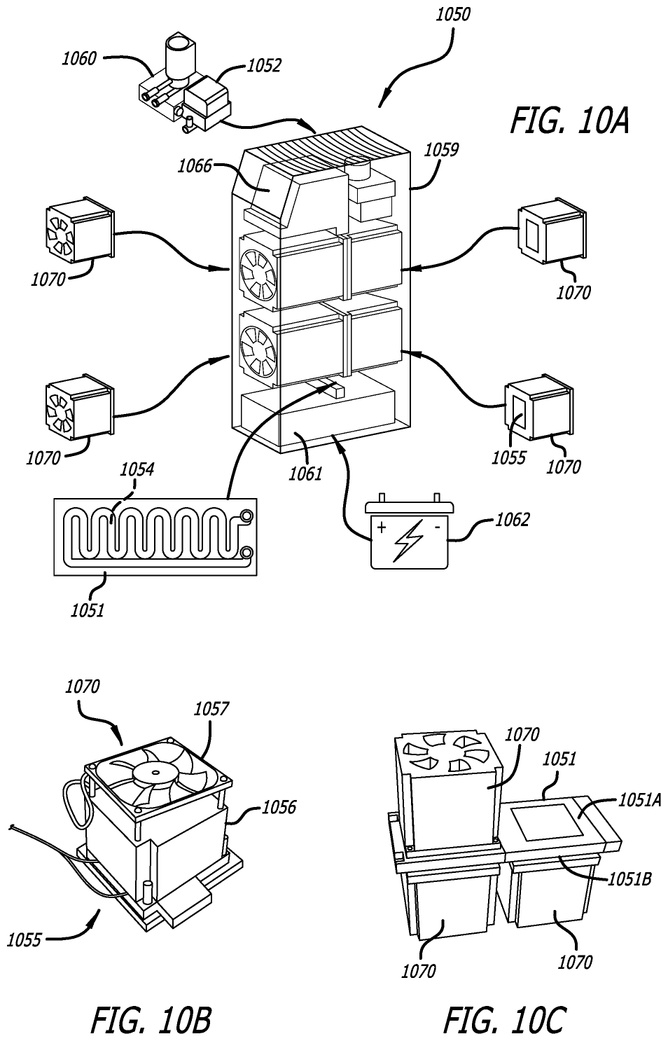

FIGS. 10A-10C illustrate a modularized temperature control (TC) module 1050 that may be employed by the either of the TTM systems 700, 900 shown and described above. The TC module 1050 can, in certain respects, resemble components of the TC module 150 described in connection with FIGS. 1-6. It will be appreciated that all the illustrated embodiments may have analogous features. Relevant disclosure set forth above regarding similarly identified features thus may not be repeated hereafter.

FIG. 10A illustrates the TC module 1050 including a housing 1059 containing the components of the TC module 1050. Also shown are some of the components of the TC module 1050 separated from TC module 1050. The TC module 1050 is generally configured to cool and/or warm a TE fluid (not shown) flowing through a number of channels 1054 of a heat exchanger 1051. A pump 1052 is configured to circulate the TE fluid between the heat exchanger 1051 and a thermal contact pad (not shown) that may resemble the pad 730 of FIGS. 7A and 9. In the illustrated embodiment, the TC module 1050 includes four TC sub-assemblies 1070 thermally coupled with the heat exchanger 1051. The TC module 1050 is configured to accommodate 1, 2, 3, or 4 TC sub-assemblies 1070. In other words, at any point in time, the TC module 1050 may include 1, 2, 3, or 4 TC sub-assemblies 1070 coupled with the heat exchanger 1051. In other embodiments, the TC module may be configured to accommodate up to 2, 3, 4, or more TC sub-assemblies 1070. The TC module 1050 includes a controller 1060 that may, in some respects, resemble the controller 760 of FIG. 8.

A power source 1061 provides electrical power to the comports of the TC module 1050. The power source 1061 may be modularized so as to accommodate a number (e.g., 1, 2, 3, 4, or more) of batteries 1062. In some embodiments, the number of batteries may correspond to the number of TC assemblies 1070. The TC module 1050 may be configured to accommodate recharging and/or replacement of one or more batteries 1062 without interrupting operation.

The TC module 1050 includes fluid chamber 1066 configured to provide a fluid reservoir for the TE fluid. The fluid chamber 1066 is open or openable at the top so that a user may add TE fluid to the fluid chamber 1066. The fluid chamber 1066 is disposed hydraulicly in line with the pump 1052, the heath exchanger 1051 and the pad.

FIG. 10B illustrates a top perspective view the TC sub-assembly 1070 including a heat sink 1056 disposed between a fan 1057 on the top side and thermoelectric device 1055 on the bottom side. The fan 1057, heat sink 1056, and the TED 1055 are fastened together to form a single unit.

FIG. 10C illustrates the arrangement of the TC assemblies 1070 coupled with the heat exchanger 1051. The heat exchanger 1051 defines a first side 1051A configured for thermally coupling TC assemblies 1070 thereto and a second side, opposite the first side, also configured for thermally coupling TC assemblies 1070 thereto. By way of example, the heat exchanger is configured for coupling two TC assemblies 1070 to each of the first side 1051A and the second side 1051B. FIG. 10C illustrates one TC assembly 1070 coupled with the first side 1051A and two TC assemblies 1070 coupled with the second side. Each TC assembly 1070 is oriented so that the TED 1055 is in thermal contact with the heat exchanger 1051 and so that the fan 1057 faces away from the heat exchanger 1051.

Referring back to FIG. 10A, the TC module 1050 defines an arrangement of the TC sub-assemblies 1070 to enable ease of coupling and decoupling of each TC sub-assembly 1070 with the heat exchanger 1051. In an exemplary implementation, the heat exchanger 1051 is disposed centrally within the housing 1059 of the TC module 1050 and each TC sub-assembly 1070 is coupled with the heat exchanger 1051. Each TC module 1050 is oriented with the fan 1057 facing a side of the housing 1059 so that the fan 1057 exhausts air away from the housing 1059.

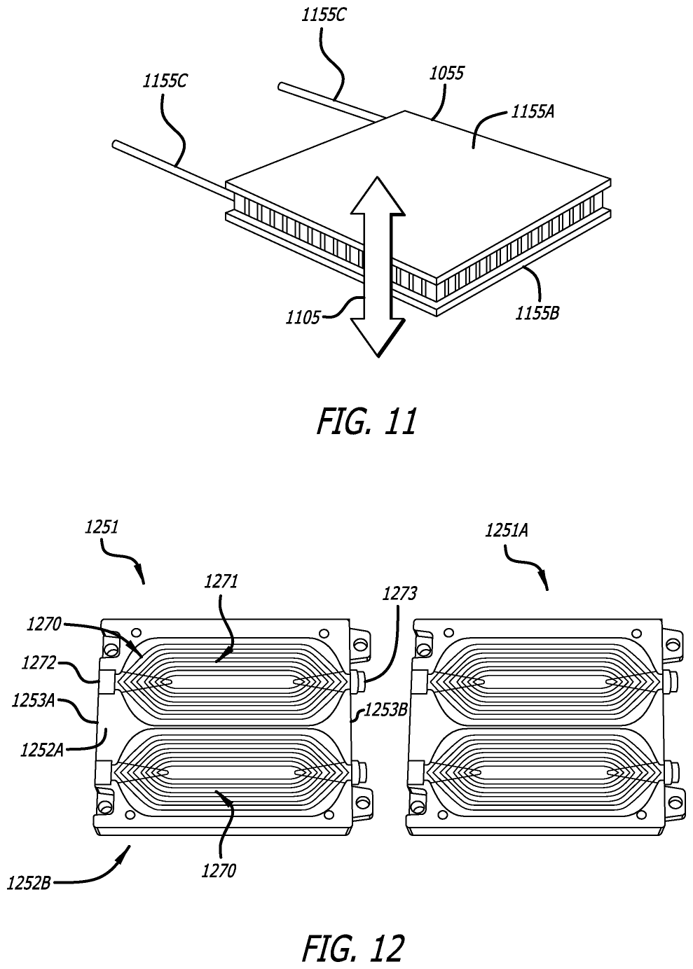

FIG. 11 is a perspective view of the TED 1055. The TED 1055 generally defines a thin rectangular shape having a first side 1155A and a second side 1155B disposed opposite the first side. In some embodiments, the TED 1055 may define a square shape having a side length of about 55 mm. The TED 1055 is generally configured to define a thermal energy exchange 1105 across the TED 1055 between the first side 1155A and the second side 1155B in accordance with an electrical power supplied to the leads 1055C. Each of the first side 1155A and the second side 1155B define a flat surface for thermal coupling the heat exchanger 1051 or the heat sink 1056 thereto.

The TED 1055 is configured to define a direction of the thermal energy exchange 1105 based on the polarity of a direct current (DC) voltage supplied to the leads 1155C. More specifically, supplying the DC voltage at a first polarity may cause a temperature of the first side 1155A to be less than a temperature of the second side 1155B. Conversely, supplying the DC voltage at an opposite second polarity causes the temperature of the first side 1155A to be greater than the temperature of the second side 1155B.

FIG. 12 illustrates another embodiment of a heat exchanger that may be employed with TC module 1050. The heat exchanger 1251 may, in some respects, resemble the features and functionality of the heat exchange 1051 of FIGS. 10A, 10C. The heat exchanger 1251 defines a first side 1252A and an opposite second side 1252B, where each side is configured to thermally couple with a TED 1055 (see FIG. 11).

The heat exchanger 1251 includes one or more sets 1270 of fluid flow channels (i.e., lumens) 1271 extending across the heat exchanger 1251 between a first port 1272 located along a first edge 1253A and a second port 1273 located along a second edge 1253B, where the second edge 1253B is disposed opposite the first edge 1253A. In the illustrated embodiment, the heat exchanger 1251 includes two sets 1270 of flow channels 1271. In other embodiments, the heat exchanger 1251 may include 1, 3, 4, or more sets 1270.

The heat exchanger 1251 is configured for coupling with additional heat exchangers 1251 such as the heat exchanger 1251A shown in FIG. 12. The heat exchangers 1251, 1251A are configured to couple with each other in an edge-to-edge fashion. More specifically, the second edge 1253B of the heat exchanger 1251 is configured to couple with a corresponding first edge 1253A of the heat exchanger 1251A. As such, each second port 1273 of the heat exchanger 1251 is configured to sealably couple with a corresponding first port 1272 of the heat exchanger 1251A.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A medical pad assembly for exchanging thermal energy with a patient, the assembly comprising:
a thermal contact pad configured for placement on the patient, the pad including a fluid compartment configured for circulation of a thermal exchange (TE) fluid therein;
a temperature control (TC) module attached to the pad, including:
a pump configured to circulate the TE fluid between the TC module and the fluid compartment; and
a thermoelectric device (TED) configured to exchange thermal energy with the TE fluid.

2. The assembly of claim 1, wherein the TC module is attached to the pad on a top side of the pad.

3. The assembly of claim 1, wherein the TC module comprises:
a heat exchanger operatively coupled with the TED, the heat exchanger configured to facilitate thermal energy transfer between a first side of the TED and the TE fluid; and
a heat sink operatively coupled with the TED on a second side of TED, the heat sink configured to facilitate thermal energy transfer between the second side of TED and the environment, the second side disposed opposite the first side.

4. The assembly of claim 1, wherein the TC module includes a power source configured to convert a facility level alternating current (AC) voltage to a direct current (DC) voltage consistent with operation of the TED.

5. The assembly of claim 1, wherein the TC module comprises a temperature controller coupled with the TED, the temperature controller configured to increase or decrease a temperature of the TE fluid.

6. The assembly of claim 5, wherein:
the TC module comprises a temperature sensor operatively coupled with the TE fluid and the temperature controller, and
the temperature controller is configured to regulate the temperature of the TE fluid in accordance with a target temperature of the TE fluid stored in a memory of the temperature controller.

7. The assembly of claim 5, wherein:
the TC module comprises a number of safety temperature sensors operatively coupled with one or more of the TE fluid, the heat exchanger, the TED, the heat sink, or the pump; and
the controller is configured to (i) compare a temperature measurement of the number of safety temperature sensors with a temperature safety limit stored in the memory and (ii) alter an electrical power supplied to the TED when the temperature measurement exceeds the temperature safety limit.

8. The assembly of claim 3, wherein the TC module is attached to the pad so that:
an outlet port of the heat exchanger is directly coupled with an inlet port of the fluid compartment, and
an outlet port of the fluid compartment is directly coupled with an inlet port of the pump.

9. The assembly of claim 1, wherein the TC module is configured for selective attachment to and detachment from the pad.

10. The assembly of claim 1, wherein the TC module includes a latch configured to selectively lock the TC module to the pad and unlock the TC module from the pad.

11. The assembly of claim 3, wherein the heat exchanger, the pump, and the fluid compartment are coupled together to define a closed fluid volume.

12. The assembly of claim 1, wherein:
the pad is one of a number of thermal contact pads having different shapes and/or sizes, and
the TC module is configured to attach singly to any one of the number of thermal contact pads.

13. The assembly of claim 1, wherein:
the assembly is transitionable from a pre-use unassembled state to an assembled state such that:
the TC module is separated from the pad in the pre-use unassembled state, and
the TC module is attached to the pad in the assembled state; and
the pad sealably contains the TE fluid within the fluid compartment when the pad is disposed in the pre-use unassembled state.

14. The assembly of claim 13, wherein:
the heat exchanger is unprimed with TE fluid in the pre-use unassembled state, and
the TE fluid flows from the fluid compartment into the heat exchanger to prime the heat exchanger upon attachment of the TC module to the pad.

15. The assembly of claim 13, wherein the TE fluid is pressurized within the fluid compartment in the pre-use unassembled state.

16-19. (canceled)

20. A medical system for exchanging thermal energy with a patient, comprising:
a thermal contact pad configured for placement on the patient, the pad including a fluid compartment configured for circulation of a thermal exchange (TE) fluid therein; and
a system module, comprising:
a heat exchanger including a number of fluid channels, the heat exchanger coupled with the pad via a fluid delivery line;
a pump configured to circulate the TE fluid between the heat exchanger and the pad;
a temperature control (TC) module operatively coupled with the heat exchanger, the TC module configured to warm and/or cool the TE fluid circulating within the heat exchanger; and
a console including one or more processors and a non-transitory computer-readable medium having stored thereon logic that, when executed by the one or more processors, causes operations of the system that include defining a temperature of the TE fluid.

21. The system of claim 20, wherein the heat exchanger is configured to couple with more than one TC module.

22. The system of claim 21, wherein the system module comprises at least two TC modules operatively coupled with the heat exchanger.

23. The system of claim 20, wherein the heat exchanger is configured to couple with:
one or more TC modules along a first side of the heat exchanger; and
one or more TC modules along a second side of the heat exchanger, the second side disposed opposite the first side.

24. The system of claim 20, wherein the TC module includes:
a thermoelectric device (TED) thermally coupled with the heat exchanger along a first side of the TED; and
a heat sink thermally coupled with the TED along a second side of the TED, the second side disposed opposite the first side.

25. The system of claim 24, wherein:
the operations include switching a polarity of an electrical power supplied to the TED between a first polarity and an opposite second polarity,
the first polarity causes the TED to supply thermal energy to the heat exchanger to increase a temperature of the TE fluid circulating with the heat exchanger, and
the second polarity causes the TED to extract thermal energy from the heat exchanger to decrease the temperature of the TE fluid circulating with the heat exchanger.

26. The system of claim 24, wherein:
the system module includes an outlet temperature sensor operatively coupled with the TE fluid exiting the heat exchanger, the outlet temperature sensor configured to provide an electrical signal to the console in accordance with a temperature of the TE fluid exiting the heat exchanger; and
the operations include:
comparing the temperature of the TE fluid exiting the heat exchanger with a target temperature for the TE fluid stored in the memory; and
as a result of the comparison, adjusting an electrical power supplied to the TED to move the temperature of the TE fluid exiting the heat exchanger toward the target temperature.

27. The system of claim 26, wherein:
the system module further includes an inlet temperature sensor operatively coupled with the TE fluid entering the heat exchanger, the input temperature sensor configured to provide an electrical signal to the console in accordance with a temperature of the TE fluid entering the heat exchanger, and
the operations include:
determining a temperature difference between the TE fluid exiting the heat exchanger and the TE fluid entering the heat exchanger; and
adjusting the electrical power supplied to the TED based on the temperature difference.

28. The system of claim 24, wherein:
the system module further includes an ambient temperature sensor configured to provide an electrical signal to the console in accordance with an ambient temperature of the patient environment, and
the operations include adjusting the electrical power supplied to the TED based on the ambient temperature.

29. The system of claim 24, wherein:
the system module further includes a safety temperature sensor operatively coupled with the TE fluid exiting the heat exchanger, the safety temperature sensor configured to provide an electrical signal to the console in accordance with a temperature of the TE fluid exiting the heat exchanger, and
the operations include:
comparing the temperature of the TE fluid exiting the heat exchanger with a safety temperature limit stored in a memory of the system module; and
as a result of the comparison, altering the electrical power supplied to the TED when the temperature of TE fluid exiting the heat exchanger exceeds the safety temperature limit.

30. The system of claim 20, wherein:
the pump is hydraulicly positioned between the pad and the heat exchanger, the pump configured to cause TE fluid to flow from the pad to the heat exchanger;
a pressure sensor is hydraulicly positioned between the pad and the pump, the pressure sensor configured to provide an electrical signal to the console in accordance with a pressure of the TE fluid within the pad; and
the operations include:
comparing the pressure of the TE fluid within the pad with a pressure limit stored in the memory; and
as a result of the comparison, altering an electrical power supplied to the pump when the pressure of the TE fluid within the pad exceeds the pressure limit.

31. The system of claim 20, wherein a power source of the system includes a battery.

30-35. (canceled)
